(19) United States
(12) Patent Application Publication
(10) Pub. No.: US 2025/0009975 A1
(43) Pub. Date: Jan. 9, 2025

(54) WIPER SEAL FOR PISTON OF DISPENSING SYRINGE

(71) Applicant: NORDSON CORPORATION, WESTLAKE, OH (US)

(72) Inventors: Matthew E. PAPPALARDO, Ewing, NJ (US); Bobby CHASSE, East Providence, RI (US)

(73) Assignee: NORDSON CORPORATION, WESTLAKE, OH (US)

(21) Appl. No.: 18/709,565

(22) PCT Filed: Feb. 16, 2023
(86) PCT No.: PCT/US2023/062704

§ 371 Date: May 13, 2024

Related U.S. Application Data
(60) Provisional application No. 63/311,258, filed on Feb. 17, 2022.

(51) Int. Cl. A61M 5/315

(52) U.S. Cl. CPC A61M 5/31513; A61M 2005/31521; A61M 2005/31523

(57) ABSTRACT

This disclosure is directed to a piston configured to be used with a dispensing syringe and a dispensing syringe having the piston. The piston has a body configured to be slidably positioned within an interior reservoir of the dispensing syringe. The body defining a wall that circumferentially surrounds the piston. The piston also has a wiper seal that extends outwardly and circumferentially from the wall. The wiper seal define a lip, a first portion, a second portion, and a junction that connects the first portion and the second portion. The second portion extends outwardly from the junction and away from the body to the lip.

### CROSS REFERENCE TO RELATED APPLICATIONS

This application is a National Stage Application of International Patent App. No. PCT/IB2023/050401, filed Jan. 17, 2023, which claims the benefit of U.S. Provisional Patent App. No. 63/311,258, filed Feb. 17, 2022, the entire disclosures of both of which are hereby incorporated by reference as if set forth in their entirety herein.

### TECHNICAL FIELD

The disclosure relates generally to the field of dispensing liquid materials, and more particularly to a wiper seal for a piston that reduces piston bounce and improves deposit consistency.

### BACKGROUND

Various types of dispensers are used in many industries for placing liquids, such as adhesives, conformal coating materials, solder paste, solder flux, and/or other similar materials, onto substrates during an assembly process. One type of liquid dispenser is a syringe-type dispenser that can have a dispenser body defining a barrel reservoir for holding a supply of liquid material to be dispensed. A dispensing tip can be coupled to the syringe at one end and the syringe can be in fluid communication with the reservoir at another end. A piston can be movably disposed in the reservoir to pressurize the liquid in the reservoir and thereby dispense a small amount of liquid from the dispensing tip and onto a substrate.

Many industrial applications need the liquid to be dispensed in precise volumes and at precise locations. To this end, liquid dispensers can include actuators for moving the piston within the reservoir in a controllable and predictable manner. For instance, pneumatic actuators can apply compressed gas, such as air, to the piston to move the piston and dispense liquid from the dispenser. Other types of actuators, such as linear actuators, can be used to control movement of the piston within the reservoir.

Dispensers can be prone to a phenomenon known as “piston bounce.” Piston bounce can refer to the accumulation of entrapped gas between the piston and the liquid. When the piston is actuated it can lurch or “bounce” on the entrapped gas before contacting the liquid within the dispenser. Piston bounce can cause retention of fluid on the reservoir wall, piston migration to the top of the reservoir, and unwanted mixing of air into the fluid. These effects of piston bounce can decrease performance of the liquid dispenser and can range from minor inconsistencies in the dispensed liquid to a tunneling of the liquid within the liquid dispenser that can require disposal of the remaining liquid.

Traditional solutions for improving performance of such liquid dispensers often attempt to balance piston bounce with liquid waste. Specifically, the piston can include or at least partially define passages such as vents, micro-vents, flow channels, and/or increased clearances to direct entrapped gas from the liquid, beyond the piston, and into an ambient environment. The micro-vents can allow air leaving the fluid to pass the piston, but can prevent the fluid from passing the piston since the fluid can clog/seal the micro-vents While relatively effective at reducing piston bounce, these passages also tend to release liquid that, in turn, creates significant waste.

There is a need for a liquid dispensing syringe and method for reducing piston bounce that effectively dispenses liquid while addressing issues such as those discussed above.

### SUMMARY

These needs are met, to a great extent, by a piston configured to be used with a dispensing syringe. The piston includes a body configured to be slidably positioned within an interior reservoir of the dispensing syringe, the body defining a wall that circumferentially surrounds the piston. The piston also includes a wiper seal that extends outwardly and circumferentially from the wall, the wiper seal defining a lip, a first portion, a second portion, and a junction that connects the first portion and the second portion. The second portion extends outwardly from the junction and away from the body to the lip.

Implementations may include one or more of the following features. The piston where in a relaxed state the second portion extends outwardly from the junction and away from the body at an angle relative to an axis that extends parallel to a longitudinal extending direction of the piston and through the junction. The angle is greater than 0°. The angle is greater than 4°. The angle is between 4° and 10°. The angle is between 0° and 45°. The first portion tapers from the junction to the wall. The junction is radially shaped with a center of curvature disposed towards the body. The wiper seal is configured to provide a liquid tight seal with an inner surface of a barrel of the dispensing syringe and to inhibit a liquid from flowing beyond the wiper seal.

One general aspect includes a dispensing syringe comprising a barrel defining an inner surface and an interior reservoir. The syringe also includes a piston slidably disposed within the interior reservoir. The piston may include a body defining a wall that circumferentially surrounds the piston and a wiper seal that extends outwardly and circumferentially from the wall. The wiper seal defining a lip, a first portion, a second portion, and a junction that connects the first portion and the second portion. The second portion extends outwardly from the junction and away from the body to the lip. An interface length of the second portion of the wiper seal contacts the inner surface of the barrel to form a liquid tight seal.

Implementations may include one or more of the following features. The dispensing syringe where the interface length of the second portion of the wiper seal that contacts the inner surface of barrel is less than two thirds of a total length of the second portion. A remaining length of the second portion defined by the total length of the second portion less the interface length does not contact the inner surface of the barrel. The junction and the first portion do not contact the inner surface of the barrel. The wiper seal has a material that is less rigid than a material of the barrel and the wiper seal is arranged in a compressed state within the barrel. The piston is removable from barrel and when removed from the wiper seal reverts to a relaxed state. In the relaxed state the second portion extends outwardly from the junction and away from the body at an angle relative to an axis that extends parallel to a longitudinal extending direction of the piston and through the junction. The angle is greater than 4°. The angle is between 4° and 10°. The first portion tapers from the junction to the wall. The junction is radially shaped with a center of curvature disposed towards the body.

Various additional objectives, advantages, and features of the disclosure will be appreciated from a review of the following detailed description of the illustrative embodiments taken in conjunction with the accompanying drawings.

### BRIEF DESCRIPTION OF THE DRAWINGS

The following detailed description is better understood when read in conjunction with the appended drawings. For the purposes of illustration, examples are shown in the drawings; however, the subject matter is not limited to the specific elements and instrumentalities disclosed. In the drawings:

FIG. 1 is a perspective view of an example dispensing syringe according to aspects of the disclosure;

FIG. 2 is an exploded perspective view of the dispensing syringe of FIG. 1;

FIG. 3A is a cross-sectional view illustrating a piston prior to insertion within a syringe barrel of FIG. 1;

FIG. 3B is an enlarged cross-sectional view similar to FIG. 3A;

FIG. 3C is an enlarged cross-sectional view illustrating an installation of the piston against a liquid within the syringe barrel of FIG. 3A;

FIG. 3D is an enlarged cross-sectional view illustrating the piston and the syringe barrel in use with a pressurized gas of FIG. 3A;

FIG. 3E is a magnified cross-section view of the encircled portion of the piston of FIG. 3A with the wiper seal in the relaxed state; and

FIG. 3F is a magnified cross-section view of the encircled portion of the piston of FIG. 3D with the wiper seal in the compressed state within the barrel.

### DETAILED DESCRIPTION OF ILLUSTRATIVE EMBODIMENTS

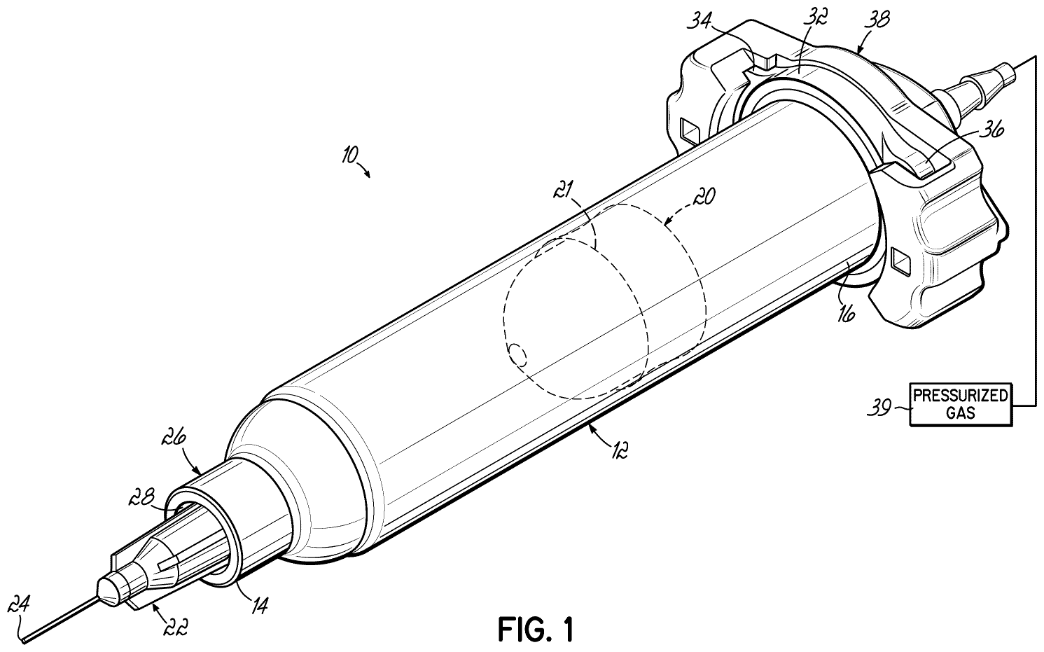

Aspects of the disclosure are directed to a piston for a dispensing syringe and to a dispensing syringe with the piston. The piston can have a wiper seal with a geometry that reduces piston bounce and improves dispensing consistency. For example, the wiper seal can extend outwardly from and circumferentially around a body of the piston. The wiper seal can define a tip, a first portion, a second portion, and a junction between the first portion and the second portion. The second portion of the wiper seal can extend outwardly from the junction and away from the body of the piston to the tip. According to this arrangement, when the piston is slidably disposed within a barrel of the dispensing piston only a fraction of the total length of the second portion of the wiper seal, for example, less than two thirds, contacts the barrel to form a liquid tight seal. The wiper seal can be configured such that any or all of the remainder of the length of the second portion, the junction, and the first portion of the wiper seal do not contact the barrel. This can reduce the surface area of the wiper seal that contacts the barrel as compared to traditional wiper seals, which can allow entrapped gas to pass more easily through the seal while preserving a liquid tight seal between the piston and the barrel. Pistons employing wiper seal geometries in accordance with aspects of this disclosure can reduce or eliminate piston bounce and dispensing consistency can be improved. These and other aspects of the disclosure are described in further detail below in reference to FIGS. 1-3F.

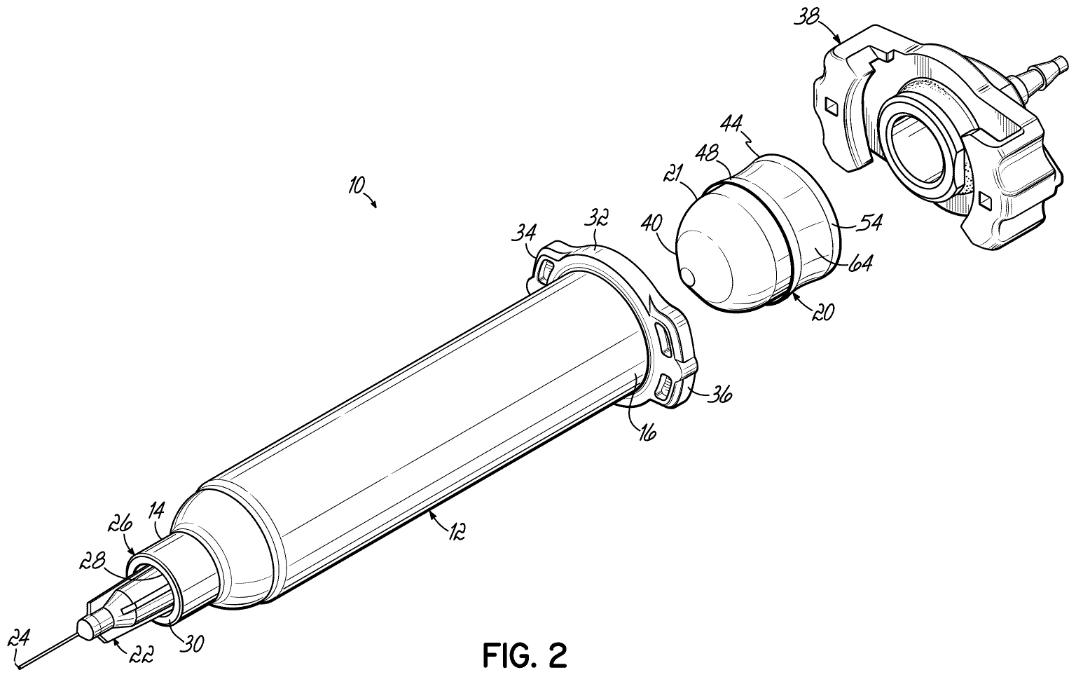

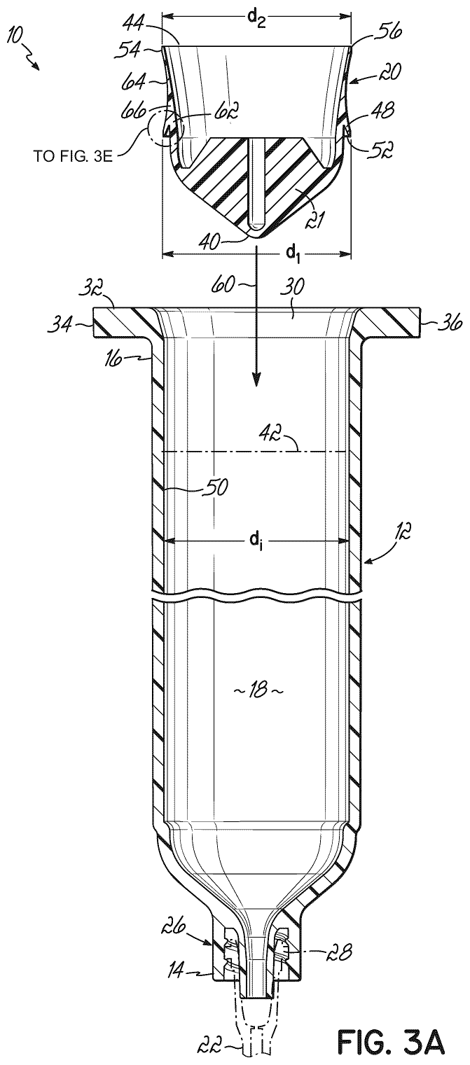

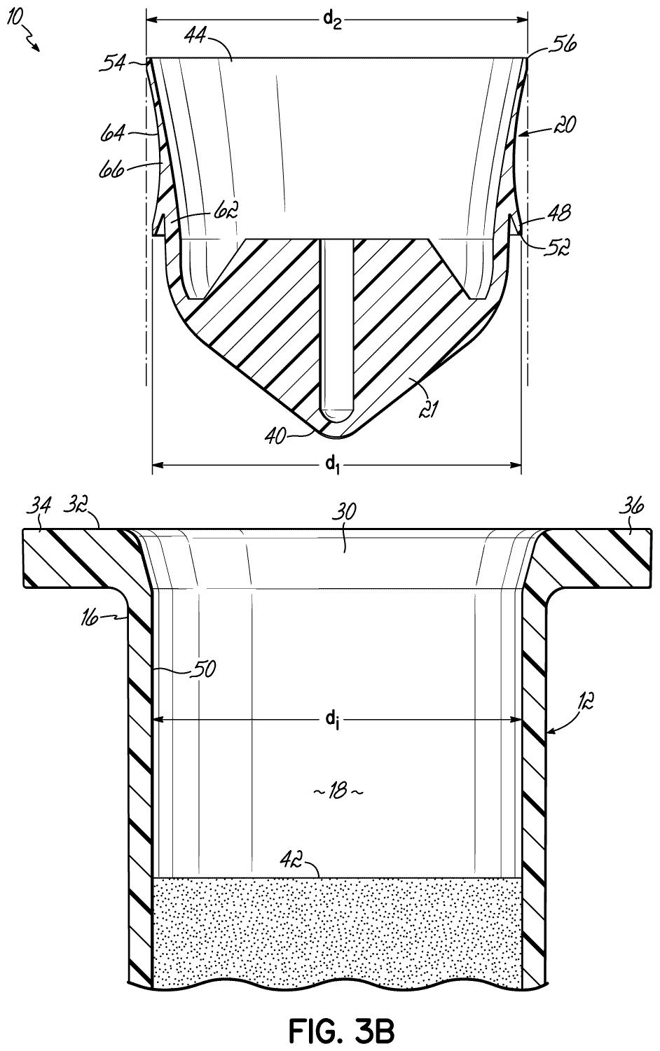

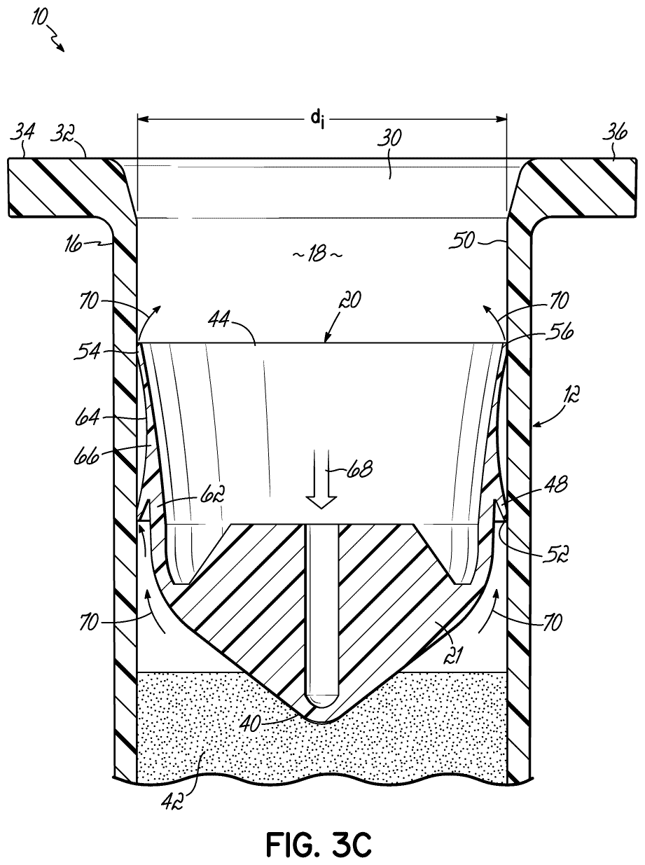

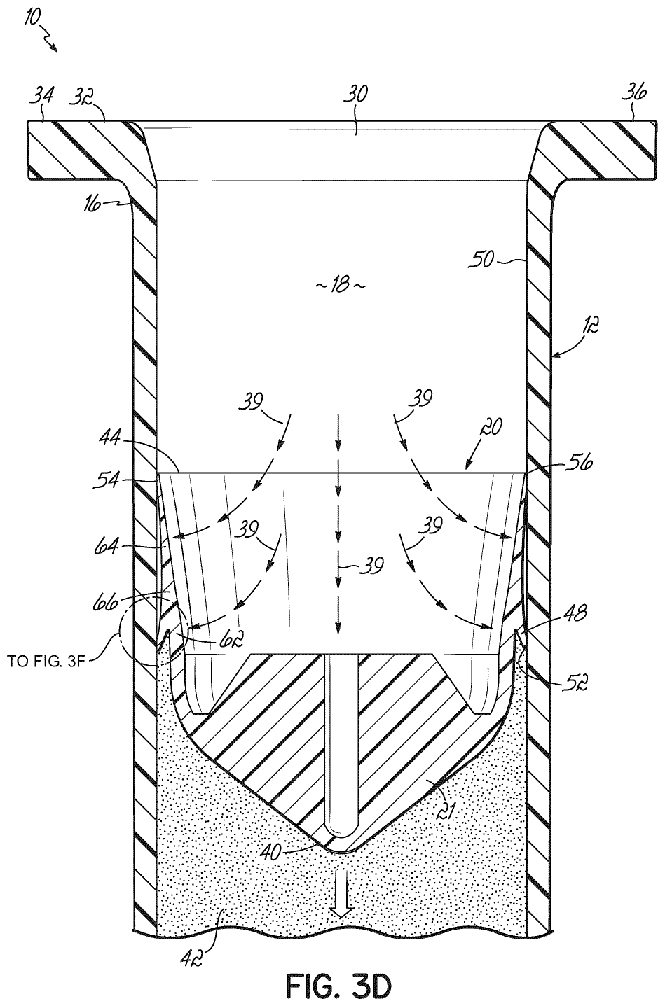

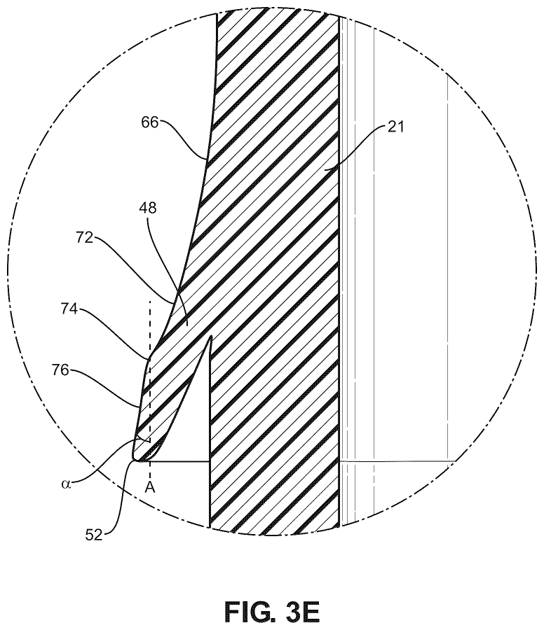

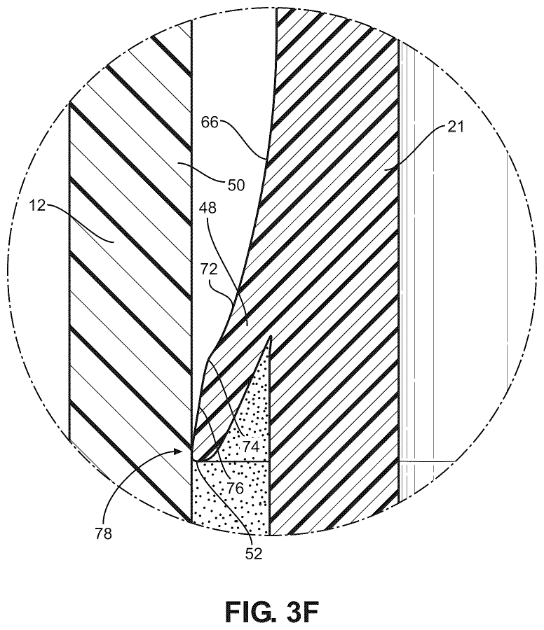

FIGS. 1-3F show aspects of an example of a dispensing syringe 10 and piston 20 of this disclosure. FIGS. 1 and 2 respectively show a perspective and an exploded-perspective view of the dispensing syringe 10. FIG. 3A shows a cross-sectional view of the dispensing syringe 10 with the piston 20 removed from the dispensing syringe 10. FIG. 3B shows an enlarged cross-sectional view of an upper portion of the dispensing syringe 10 with the piston 20 removed from the dispensing syringe 10. FIG. 3C shows an enlarged cross-sectional view of an upper portion of the dispensing syringe 10 with the piston 20 slidably disposed in the dispensing syringe 10. FIG. 3D shows an enlarged cross-sectional view of an upper portion of the dispensing syringe 10 with the piston 20 slidably disposed in the dispensing syringe 10 and fully engaged with a dispense fluid. FIG. 3E shows a magnified view of the piston 20 in a relaxed state from the encircled region of FIG. 3A. FIG. 3F shows a magnified view of the piston 20 in a compressed state from the encircled region of FIG. 3D.

The dispensing syringe 10 can include a generally elongate barrel 12 having a first end 14 for dispensing liquid material therefrom, and a second end 16 opposite the first end 14. The barrel 12 can define an interior reservoir 18 (as shown for example in FIG. 3A) for containing the liquid material to be dispensed from the first end 14. The dispensing syringe 10 can include a piston 20 having a body 21 that can be slidably disposed within the interior reservoir 18 and is slidably movable therein between the first end 14 and the second end 16 to dispense liquid material from the first end 14 as the piston 20 moves in a direction toward the first end 14. The dispensing syringe 10 can include a dispensing tip 22 that can be removably coupled to the first end 14 of the barrel 12 for communication with the interior reservoir 18 such that liquid material can be dispensed from an outlet 24 of the dispensing tip 22. According to an example embodiment, the barrel 12 can be sized to contain 55 cc of liquid. It will be appreciated, however, that the barrel 12 can be any size for containing a desirable volume of liquid. Furthermore, the larger syringe barrels can also be referred to as “cartridges” or “cartridge barrels.” In this respect, the terms “syringe” and “cartridge” are not intended to limit the disclosure to any particular volume or barrel.

The first end 14 of the barrel 12 can include a first connector 26 adapted to receive a corresponding second connector 28 provided on the dispensing tip 22, such that the dispensing tip 22 can be coupled to the first end 14 of the barrel 12. The second end 16 of the barrel 12 can include an opening 30 (as shown for example in FIG. 3A) and can have a radially outwardly extending flange 32. The flange 32 can include a pair of oppositely disposed tabs or ears 34, 36 that can extend radially outward from the flange 32, in opposite directions, to facilitate securing an adapter 38 for coupling the dispensing syringe 10 to an actuator, such as a source of a pressurized gas 39. The barrel 12, the dispensing tip 22, the first connector 26, the adapter 38, and portions of the piston 20 are described in additional detail in U.S. Pat. No. 9,958,067, the disclosure of which is hereby incorporated by reference herein in its entirety.

An example embodiment of the piston 20 can include a distal end 40 configured to force a liquid material 42 (as shown for example in FIG. 3B) through the first end 14 of the barrel 12 and into the dispensing tip 22. With reference to FIG. 2, the distal end 40 of the piston 20 can mate with the second end 16 of the barrel 12 to force the liquid material 42 into the dispensing tip 22. A proximal end 44 of the piston 20 opposite the distal end 40 can be exposed to the pressurized gas 39, whereby the piston 20 can be actuated in a direction from the second end 16 of the barrel 12 toward the first end 14 of the barrel 12. For example, the pressurized gas 39 can be pressurized air provided in a typical shop setting, manufacturing setting, and/or the like. Alternatively, the piston 20 can be actuated manually, with any other actuator, and/or the like.

As shown in FIG. 3A, the piston 20 can include a first wiper seal 48 that can extend radially outwardly from the piston 20 and can engage an inner surface 50 of the barrel 12 to seal the first wiper seal 48 against the inner surface 50. The first wiper seal 48 can be positioned on the piston 20 between the distal end 40 and the proximal end 44. In an example embodiment, the first wiper seal 48 can be a flange or skirt and can extend circumferentially around the body 21 of the piston 20. The first wiper seal 48 can have an interference fit with the inner surface 50 of the barrel 12 that can ensure that the first wiper seal 48 conforms to the inner surface 50 of the barrel 12 and can radially compensate for any shape irregularities of the first wiper seal 48 and/or of the inner surface 50 of the barrel 12. In this regard, the first wiper seal 48 can be structured and/or configured with the interference fit with the inner surface 50 of the barrel 12 that can ensure that the first wiper seal 48 conforms to the inner surface 50 of the barrel 12 and can radially compensate for any shape irregularities of the first wiper seal 48 and/or of the inner surface 50 of the barrel 12.

As shown in resolution provided in the magnified view of FIGS. 3E and 3F, the first wiper seal 48 can include a first lip 52, a first portion 72, a second portion 76, and a junction 74 that can connect the first portion 72 and the second portion 76. The first portion 72 can taper from the junction 74 to a wall 66 of the piston 20. The junction 74 can be radially shaped and can have a center of curvature disposed towards the body 21 of the piston 20. The second portion 76 can extend outwardly from the junction 74 and away from the body 21 to the first lip 52. For example, when the piston 20 is in a relaxed state outside of the barrel 12 as shown in FIG. 3E, the second portion 76 can extend outwardly from the junction 74 and away from the body 21 at an angle α relative to an axis A that extends parallel to a longitudinal extending direction of the piston 20 and through the junction 74. The magnitude of the angle α can be selected based upon material properties, for example a rigidity, of the first wiper seal 48 and/or on the difference between a first outer diameter d₁ of the first lip 52 and an inner diameter d₁ of the interior reservoir 18. In embodiments, the angle α can be greater than 0°, greater than 4°, greater than 6°, greater than 8°, greater than 10°, greater than 12°, greater than 14°, greater than 16°, greater than 18°, greater than 20°, greater than 24°, greater than 28°, and/or greater than 32° among other possibilities. In embodiments, the angle α can be between 0° and 45°, between 4° and 10°, between 10° and 15°, between 15° and 25°, among other possibilities. In embodiments, the angle α can be less than 4°, less than 6°, less than 8°, less than 10°, less than 12°, less than 14°, less than 16°, less than 18°, less than 20°, less than 24°, less than 28°, and/or less than 32° among other possibilities. In aspects, the first portion 72 radially extends from the wall 66 outwardly at an angle relative to the axis A that extends parallel to a longitudinal extending direction of the piston 20 and through the junction 74 that is greater than the angle α. In aspects, the first portion 72 radially extends from the wall 66 outwardly at an angle relative to the axis A that extends parallel to a longitudinal extending direction of the piston 20 and through the junction 74 that is less than the angle α. In aspects, the first portion 72 may have a generally planar exterior surface between the wall 66 and the junction 74. In aspects, the second portion 76 may have a generally planar exterior surface between the junction 74 and the first lip 52.

As shown in FIG. 3F, the piston 20 can be slidably disposed within the barrel 12 of the dispensing syringe 10 and the first wiper seal 48 can be compressed inwardly in a compressed state. In the compressed state, the first wiper seal 48 can engage the inner surface 50 of the barrel 12. For example, the first wiper seal 48 can be formed of a material that is less rigid than a material forming of the inner surface 50 of the barrel such that the first wiper seal 48 is compressed in the compressed state when slidably disposed within the barrel 12.

Due to the geometry of the first wiper seal 48, i.e., the above-described structural relationship between the first lip 52, the first portion 72, the junction 74, and/or the second portion 76, the first wiper seal 48 can contact the inner surface 50 of the barrel 12 at an interface 78 defined by only a fraction of the surface area of the first wiper seal 48. For example, in embodiments only the first lip 52 and the second portion 76 of the first wiper seal 48 form the interface 78 with the inner surface 50 of the barrel 12. That is the junction 74 and the first portion 72 do not contact the inner surface 50 of the barrel 12. In embodiments, some of the second portion 76 also does not contact the inner surface 50 of the barrel 12. For example, the second portion 76 can have an interface length that contacts the inner surface 50 of the barrel 12 and a remainder of the total length of the second portion 76 does not contact the inner surface 50 of the barrel 12. In embodiments, the interface length can be less than two thirds of the total length of the second portion 76. In embodiments, the interface length can be less than half of the total length of the second portion 76. In embodiments, the interface length can be 10%, 20%, 30%, 40%, 50%, 60%, and/or 70% of the total length of the second portion 76. In embodiments, the interface length can be 10%-20, 20%-30, 30%-40, 40%-50, 50%-60, and/or 60%-70% of the total length of the second portion 76. Because only a fraction of the first wiper seal 48 can contact the inner surface 50 of the barrel 12, piston bounce can be reduced or eliminated since entrapped gas 70 can more easily escape through the first wiper seal 48. This was an unexpected result and is a result of the construction of at least the first wiper seal 48, the first lip 52, the first portion 72, the second portion 76, and/or the junction 74 as disclosed. Additionally, the geometry of the first wiper seal 48 provides a liquid tight seal with the inner surface 50 of the barrel 12. These features combine to improve dispensing consistency from the dispensing syringe 10.

Additionally, the piston 20 can include a second wiper seal 54 that can be positioned at the proximal end 44 on the piston 20. The second wiper seal 54 can be formed generally of the same material as the first wiper seal 48 and can have a second lip 56 that can engage the inner surface 50 of the barrel 12. In embodiments (not shown), the second wiper seal 54 can be provided with or without a first portion, a second portion, and a junction described above in reference to the first wiper seal 48. With respect to the use of the terms “distal” and “proximal,” it will be appreciated that such directions and/or locations are intended to describe relative locations longitudinally along example embodiments of the dispensing syringe 10. It is not intended that these terms or any other spatial references limit the disclosure to any of the example embodiments described herein.

As shown in FIGS. 3A and 3B, the inner surface 50 of the interior reservoir 18 can be generally cylindrical and can have an inner diameter d₁. The first lip 52 and the second lip 56 can each have respective first and second outer diameters d₁, d₂ in a relaxed state when the piston 20 is disposed outside of the interior reservoir 18. According to an example embodiment, in the relaxed state the first and second diameters d₁, d₂ can each be larger than the inner diameter d₁ prior to insertion into the interior reservoir 18. As such, when the piston 20 is inserted into interior reservoir 18, as indicated by arrow 60, at least the first lip 52 and the second lip 56 can compress into a compressed state and can form an interference fit with the inner surface 50 of the barrel. Moreover, the second diameter d₂ can be at least as large as the first diameter d₁. According to an example embodiment, the second diameter d₂ can be larger than the first diameter d₁ as shown in the relaxed state.

The piston 20 can include a generally rigid portion 62 and an elastic portion 64. In aspects, the generally rigid portion 62 is more rigid than the elastic portion 64. The first wiper seal 48 can be positioned on the generally rigid portion 62 of the piston 20 and the second wiper seal 54 can be positioned on the elastic portion 64 of the piston 20. The generally rigid portion 62 can maintain the first wiper seal 48 with a generally constant shape and size regardless of whether or not pressure is applied within the body 21 of the piston 20. In contrast, the elastic portion 64 can elastically deform under the influence of pressure applied within the body 21 and can act upon the second wiper seal 54. As described herein, the term “elastic” generally means that the elastic portion 64 of the piston 20 is flexible such that the elastic portion 64 expands under the influence of a force and then contracts when that force is removed or reduced. In addition, the elastic portion 64 of the piston 20 may be more flexible than the rigid portion 62. The material of construction for the piston 20 need not be a material having significant elasticity itself, but can be a flexible material formed as in the present case so as to result in the piston 20 having one or more relatively rigid portions and or more relatively flexible, elastic portions.

According to an example embodiment, the generally rigid portion 62 and the elastic portion 64 can be formed of a unitary construction from a same material, such as low-density polyethylene. The elastic portion 64 can be created by varying the geometry of the piston 20 such that the elastic portion 64 is more elastic than the generally rigid portion 62. As shown more clearly in FIG. 3B, the wall 66 of the piston 20 tapers proximally from the first wiper seal 48 toward the second wiper seal 54 to form the elastic portion 64. Because the wall 66 is thinner than the generally rigid portion 62, the wall 66 is more elastic than the generally rigid portion 62. It will be appreciated, however, that other shapes or various materials can be used alone or in combination for forming the generally rigid portion 62 and the elastic portion 64. For example, the piston 20 can be formed from two or more materials having different material properties related to elasticity. In alternative embodiments, the piston 20 can be provided without the elastic portion 64 and the piston 20 can be constructed with a generally consistent geometry from the proximal end 44 to the distal end 40 of the piston 20. In such embodiments, the elasticity of the piston 20 can be similar from the proximal end 44 to the distal end 40 of the piston 20. In both embodiments, as discussed previously the first wiper seal 48 can inhibit the liquid material 42 from passing proximally beyond the first wiper seal 48, while the second wiper seal 54 can inhibit pressurized gas 39 from passing distally beyond the second wiper seal 54. In other words, the first wiper seal 48 liquidly seals against the inner surface 50, and the second wiper seal 54 fluidly seals against the inner surface 50 at least when the proximal end 44 of the piston 20 receives pressurized gas 39.

As shown in FIG. 3C, the piston 20 can be inserted into the interior reservoir 18 and against the liquid material 42, as indicated by arrow 68, with or without the influence of the pressurized gas 39. Entrapped gas 70, such as air between the liquid material 42 and the piston 20, can flow past each of the first wiper seal 48 and the second wiper seal 54 and evacuate the interior reservoir 18, as indicated by arrows in FIG. 3C. According to an example embodiment, neither the first wiper seal 48 nor the second wiper seal 54 include passages, such as vents, micro-vents, flow channels, and/or increased clearances for encouraging the flow of entrapped gas 70 from the interior reservoir 18. Rather, the entrapped gas 70 can escape past the first wiper seal 48 and the second wiper seal 54 as the volume containing the liquid material 42 within the interior reservoir 18 decreases. Alternatively, the first wiper seal 48 can include passages (not shown), such as vents, micro-vents, flow channels, and/or increased clearances in combination with the elastic portion 64 to further encourage the flow of entrapped gas 70. The passages (not shown) provide for the flow of entrapped gas 70, but generally fill with the liquid material 42 once the entrapped gas 70 is removed to dynamically seal the liquid material 42 within the interior reservoir 18. Once the entrapped gas 70 is generally removed from between the piston 20 and the liquid material 42, the distal end 40 of the piston 20 can be positioned against the liquid material 42 and the first wiper seal 48 can seal the liquid material 42 within the barrel 12.

As shown in FIG. 3D, the pressurized gas 39 can move distally along the barrel 12 until being received by the proximal end 44 of the piston 20. In turn, the pressurized gas 39 can act on the piston 20 in two distinct ways. First, the pressurized gas 39 can force the distal end 40 against the liquid material 42 for discharging the liquid material 42 from the dispensing syringe 10. Second, the pressurized gas 39 can be applied to the elastic portion 64 to expand the elastic portion 64 to force the second wiper seal 54 against the inner surface 50. According to an example embodiment, the second wiper seal 54 can compress against the inner surface 50. The second wiper seal 54 can distally expand to contact the inner surface 50 with an increased pressure and an increased surface area to fluidly seal against the pressurized gas 39. Once flow of the pressurized gas 39 stops, the elastic portion 64 can contract and the second wiper seal 54 can contact the inner surface 50 with a reduced pressure and a reduced surface area, as shown in FIG. 3C. In this respect, the type of seal provided by the second wiper seal 54 can vary depending on the application of the pressurized gas 39. Specifically, the second wiper seal 54 can allow the entrapped gas 70 to pass, but can fluidly seal against the pressurized gas 39 while discharging the liquid material 42. Alternatively and according to another example embodiment, the elastic portion 64 can force the second wiper seal 54 against the inner surface 50 with the increased pressure, but without contacting the inner surface 50 with increased surface area. For example, the elastic portion 64 can deform via the pressurized gas 39; however, the second wiper seal 54 can simply be forced against the inner surface 50 without expanding distally. Thus, an alternative embodiment of a second wiper seal 54 can be generally rigid and fluidly seal against the pressurized gas 39 with the increased pressure alone. In either embodiment, the second wiper seal 54 can inhibit the pressurized gas 39 from incidentally passing beyond the first wiper seal 48 and between the piston 20 and liquid material 42 as entrapped gas 70. Thus, the distal end 40 is maintained against the liquid material 42 as the piston 20 moves through the interior reservoir 18. Furthermore, an example embodiment of the second wiper seal 54 does not include any passages for releasing entrapped gas 70 along the piston 20 to an ambient environment.

In aspects, the disclosure has set forth a liquid dispensing syringe and method for reducing piston bounce that includes a barrel and a piston. This was an unexpected result and is a result of the construction of at least the first wiper seal 48, the first lip 52, the first portion 72, the second portion 76, and/or the junction 74 as disclosed. The barrel defines an interior reservoir having an inner surface. The piston is disposed within the interior reservoir and has a proximal end and an elastic portion configured to expand under the influence of a pressurized gas. A first circumferentially extending wiper seal is positioned on the piston. The first wiper seal having a flexible seal wiper with sealing surface that is set at an angle which when the first wiper seal is reduced to the inside diameter of the barrel, makes the seal surface parallel and flush with the inner inside diameter of the barrel to liquidly seal against the inner surface.

In aspects, the disclosure has set forth an implementation of a piston with a flared wiper for use in contact with a barrel/container that enables air present in the fluid to bypass the wiper, but does not permit air outside the wiper to bypass the seal and get into the fluid. This was an unexpected result and is a result of the construction of at least the first wiper seal 48, the first lip 52, the first portion 72, the second portion 76, and/or the junction 74 as disclosed. Compared to current pistons, the flared surface improves interference with the barrel at the sealing point while reducing the force required to move the piston. This was an unexpected result and is a result of the construction of at least the first wiper seal 48, the first lip 52, the first portion 72, the second portion 76, and/or the junction 74 as disclosed. This combination of properties enables air to pass over the wiper but prevents fluids from passing, causing entrapped air in the fluid to vent past the piston during normal operation. This was an unexpected result and is a result of the construction of at least the first wiper seal 48, the first lip 52, the first portion 72, the second portion 76, and/or the junction 74 as disclosed.

In aspects, the disclosure includes a method of manufacture where wipers smear, deform, and/or the like on ejection due to plastic's pliable nature when hot. The combination of design geometry, Polyethylene's tendency to stick, and a surface texture on the wiper causes a reproducible flare as described herein and illustrated.

In aspects of the disclosure, the various disclosed features provide improved piston performance. This was an unexpected result and is a result of the construction of at least the first wiper seal 48, the first lip 52, the first portion 72, the second portion 76, and/or the junction 74 as disclosed. In aspects of the disclosure, the various disclosed features provide less bounce. This was an unexpected result and is a result of the construction of at least the first wiper seal 48, the first lip 52, the first portion 72, the second portion 76, and/or the junction 74 as disclosed.

In aspects of the disclosure, the various disclosed features provide better wall wipe. This was an unexpected result and is a result of the construction of at least the first wiper seal 48, the first lip 52, the first portion 72, the second portion 76, and/or the junction 74 as disclosed. In trials with a highly bounce-prone fluid, roughly ⅔ of the current pistons bounced. In the same trial, the flared piston had little or no bounce. This was an unexpected result and is a result of the construction of at least the first wiper seal 48, the first lip 52, the first portion 72, the second portion 76, and/or the junction 74.

It will be appreciated that the foregoing description provides examples of the disclosed machine. However, it is contemplated that other implementations of the disclosure can differ in detail from the foregoing examples. All references to the disclosure or examples thereof are intended to reference the particular example being discussed at that point and are not intended to imply any limitation as to the scope of the disclosure more generally. All language of distinction and disparagement with respect to certain features is intended to indicate a lack of preference for those features, but not to exclude such from the scope of the disclosure entirely unless otherwise indicated. All methods described herein can be performed in any suitable order unless otherwise indicated herein or otherwise clearly contradicted by context.

1. A piston configured to be used with a dispensing syringe, the piston comprising:
a body configured to be slidably positioned within an interior reservoir of the dispensing syringe, the body defining a wall that circumferentially surrounds the piston; and
a wiper seal that extends outwardly and circumferentially from the wall, the wiper seal defining a lip, a first portion, a second portion, and a junction that connects the first portion and the second portion,
wherein the second portion extends outwardly from the junction and away from the body to the lip.

2. The piston of claim 1, wherein in a relaxed state the second portion extends outwardly from the junction and away from the body at an angle relative to an axis that extends parallel to a longitudinal extending direction of the piston and through the junction.

3. The piston of claim 2, wherein the angle is greater than 0°.

4. The piston of claim 2, wherein the angle is greater than 4°.

5. The piston of claim 2, wherein the angle is between 4° and 10°.

6. The piston of claim 2, wherein the angle is between 0° and 45°.

7. The piston of claim 1, wherein the first portion is structured to taper from the junction to the wall.

8. The piston of claim 1, wherein the junction is radially shaped with a center of curvature disposed towards the body.

9. The piston of claim 1, wherein the wiper seal is configured to provide a liquid tight seal with an inner surface of a barrel of the dispensing syringe and to inhibit a liquid from flowing beyond the wiper seal.

10. A dispensing syringe comprising:
a barrel defining an inner surface and an interior reservoir; and
a piston slidably disposed within the interior reservoir, the piston comprising:
a body defining a wall that circumferentially surrounds the piston; and
a wiper seal that extends outwardly and circumferentially from the wall, the wiper seal defining a lip, a first portion, a second portion, and a junction that connects the first portion and the second portion,
wherein the second portion extends outwardly from the junction and away from the body to the lip, and
wherein an interface length of the second portion of the wiper seal contacts the inner surface of the barrel to form a liquid tight seal.

11. The dispensing syringe of claim 10, wherein the interface length of the second portion of the wiper seal that contacts the inner surface of the barrel is less than two thirds of a total length of the second portion.

12. The dispensing syringe of claim 11, wherein a remaining length of the second portion defined by the total length of the second portion less the interface length does not contact the inner surface of the barrel.

13. The dispensing syringe of claim 12, wherein the junction and the first portion do not contact the inner surface of the barrel.

14. The dispensing syringe of claim 10, wherein the wiper seal has a material that is less rigid than a material of the barrel and the wiper seal is arranged in a compressed state within the barrel.

15. The dispensing syringe of claim 14, wherein the piston is removable from barrel and when removed from the wiper seal is configured to revert to a relaxed state.

16. The dispensing syringe of claim 15, wherein in the relaxed state the second portion extends outwardly from the junction and away from the body at an angle relative to an axis that extends parallel to a longitudinal extending direction of the piston and through the junction.

17. The piston of claim 16, wherein the angle is greater than 4°.

18. The piston of claim 17, wherein the angle is between 4° and 10°.

19. The piston of claim 10, wherein the first portion is structured to taper from the junction to the wall.

20. The piston of claim 19, wherein the junction is radially shaped with a center of curvature disposed towards the body.
